(19) United States
(12) Plant Patent Application Publication
(10) Pub. No.: US 2006/0064791 P1
(43) Pub. Date: Mar. 23, 2006

(54) DENDROBIUM PLANT NAMED 'YFY-HS1'

(76) Inventor: Lih-Hwa Wang, Taipei (TW)

Correspondence Address: Mathews, Collins, Shepherd & Gould, P.A.;Suite 306, 100 Thanet Circle, Princeton, NJ, 08540-3674, US

(21) Appl. No.: 10/943,358

(22) Filed: Sep. 17, 2004

(51) Int. Cl. A01H 5/00

(52) U.S. Cl. PLT/311

(57) ABSTRACT

A new and distinct variety of Dendrobium plant named ‘YFY-HS1’ is provided. The plant is characterized by having a great growth vigor and the extract of “DCMPbL6, 7D2H2” defined in U.S. patent Ser. No. 10/648,651.

### FIELD OF THE INVENTION

This invention relates to a new and distinct variety of orchid plant, and more particularly to a hybrid of the Genus Dendrobium.

### BACKGROUND AND DESCRPITION OF THE INVENTION

Nowadays, the Genus Dendrobium is considered to be one of the most precious Chinese herbs for treating ophthalmic defects. The Genus Dendrobium is a member of the Family Orchidaceae. The stem of the Dendrobium plant is the mainly medicinal part. Some Chinese medical codices disclose that the Dendrobium plant is the curative for some illnesses such as salivary defects, stomach defects, and ophthalmic defects.

It has been proved in U.S. patent Ser. No. 10/648,651 that an extract of “DCMPbL6, 7D2H2” from the Dendrobii Caulis plant has the curative effects for the ophthalmic defects. However, since the Dendrobii Caulis has a slow growth property and its relevant resistance to the environment is poor, a plant having a fast growth nature and an abundant content of the extract of “DCMPbL6, 7D2H2” is desirous. After a long-term research, it's found that D. huoshanese C. Z. Tang et S. J. Cheng has the extract of “DCMPbL6, 7D2H2” but a slow growth rate, and D. tosaense Makino does not have the extract of “DCMPbL6, 7D2H2” but a fast growth ability. In order to obtain a plant having a great growth vigor and the extract of “DCMPbL6, 7D2H2”, the applicant tried to cross D. huoshanese C. Z. Tang et S. J. Cheng (being the patrilineage) with D. tosaense Makino (being the matrilineage). Accordingly, the Dendrobium plant named “YFY-HS1” is obtained.

### BRIEF DESCRIPTION OF THE DRAWINGS

FIG. 1 shows the flower of the D. huoshanese C. Z. Tang et S. J. Cheng;

FIG. 2 shows the flower of the D. tosaense Makino;

FIG. 3 shows the flower of the “YFY-HS1”;

FIG. 4 shows the growth types of the matured D. huoshanese C. Z. Tang et S. J. Cheng, the matured D. tosaense Makino and the matured “YFY-HS1”;

FIG. 5 shows the HPLC profile of the D. tosaense Makino;

FIG. 6 shows the HPLC profile of the D. huoshanese C. Z. Tang et S. J. Cheng; and

FIG. 7 shows the HPLC profile of the “YFY-HS 1”.

### SPECIFIC DESCRIPTION OF THE PLANT

Please refer to Table 1, which shows some characteristics of the D. huoshanese C. Z. Tang et S. J. Cheng, the D. tosaense Makino and the “YFY-HS1”.

TABLE 1
The partial characteristics of the D. huoshanese C. Z. Tang et
S. J. Cheng, the D. tosaense Makino and the “YFY-HS1”.

| | | | | D. huoshanense | |
|---|---|---|---|---|---|
| | | D. tosaense | | C. Z. Tang et | |
| | | Makino (the | | S. J. Cheng (the | |
| Species | YFY-HS1 | matrilineage) | | patrilineage) | |
| Stem Height | 20-35 cm | 25-45 | cm | 8-12 | cm |
| Dry weight/per plant | 2-4 g | 5-8 | g | 0.6-1.5 | g |
| Flower shape^{#} | Similar to the | | | | |
| | patrilineal | | | | |
| Growth type⁺ | Similar to the | | | | |
| | matrilineal | | | | |
| HPLC Profile* | Similar to the | | | | |
| | patrilineal | | | | |

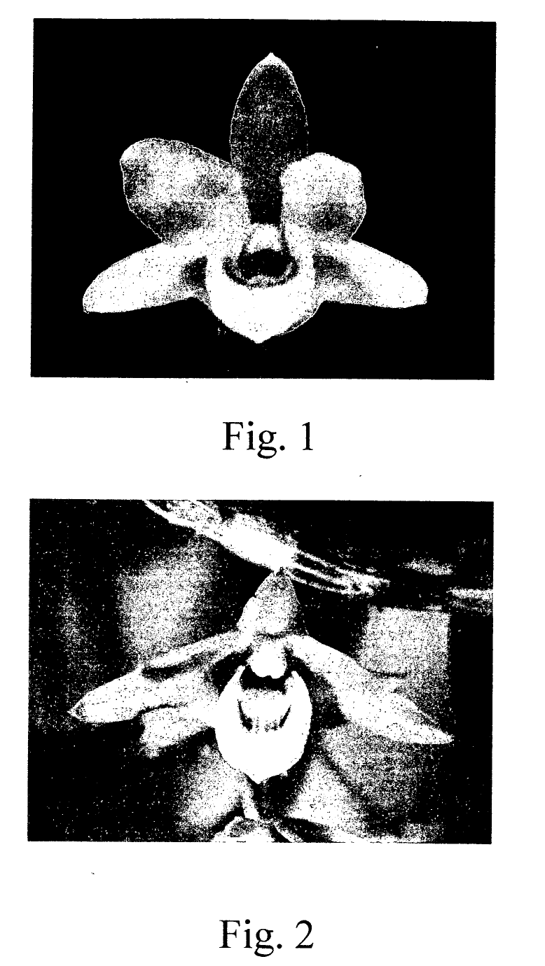

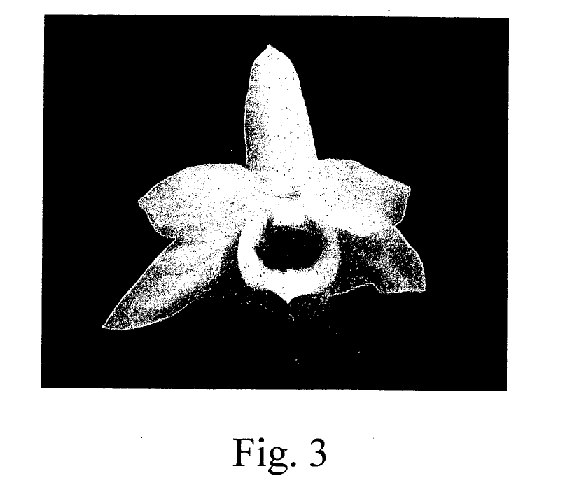

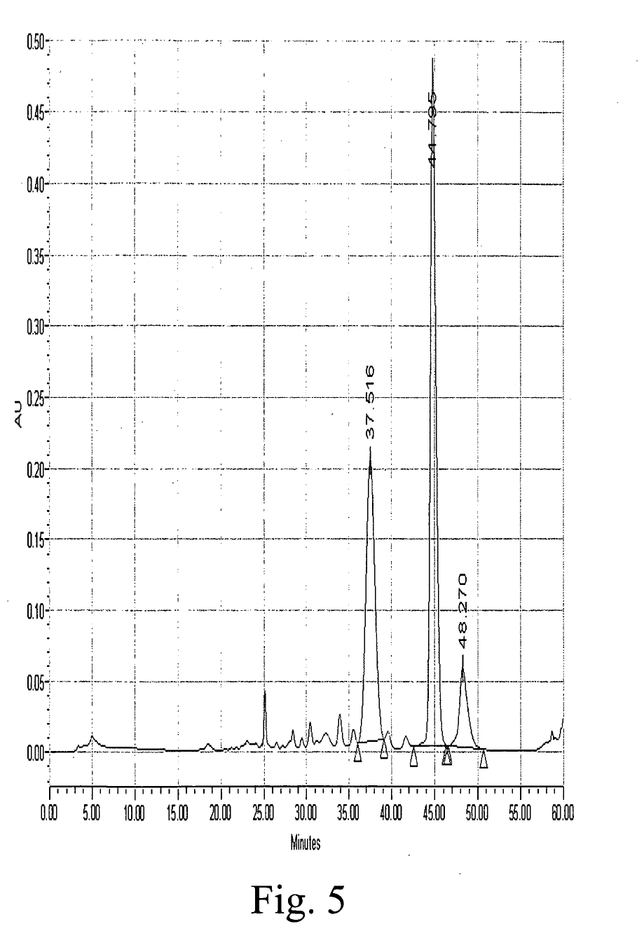

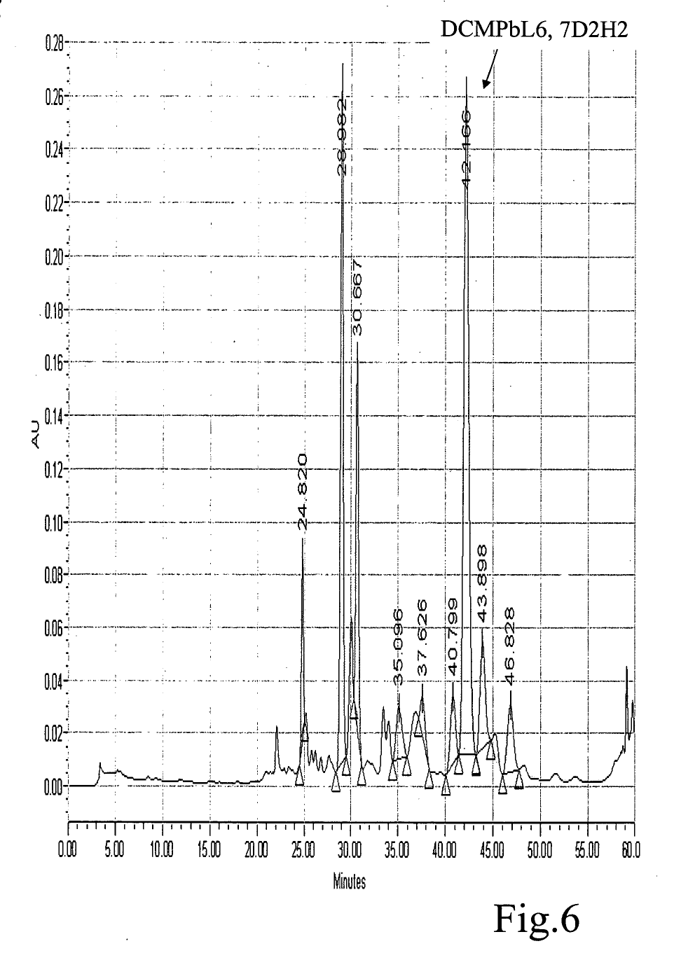

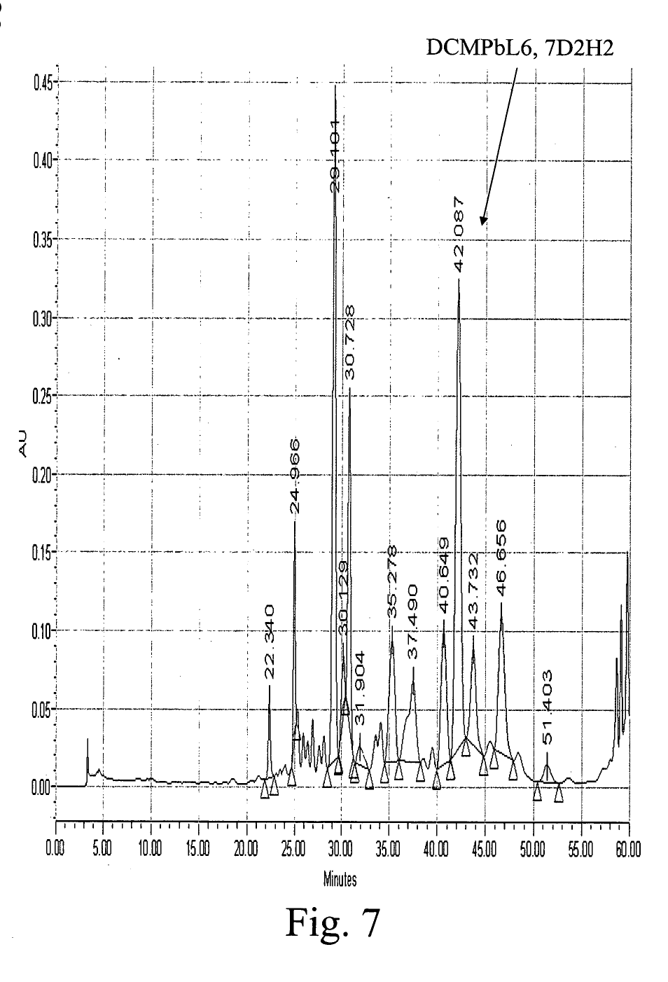

^{#,+}The detailed could be found in the follow-up FIGS. 1-4.
*The detailed could be found in the follow-up FIGS. 5-7.

Please refer to FIGS. 1-3, which respectively show the flowers of the D. huoshanese C. Z. Tang et S. J. Cheng, the D. tosaense Makino and the “YFY-HS1”. As shown in FIGS. 1-3, the flower shape of the “YFY-HS1” is similar to that of the D. huoshanese C. Z. Tang et S. J. Cheng, but the relevant color layouts are different.

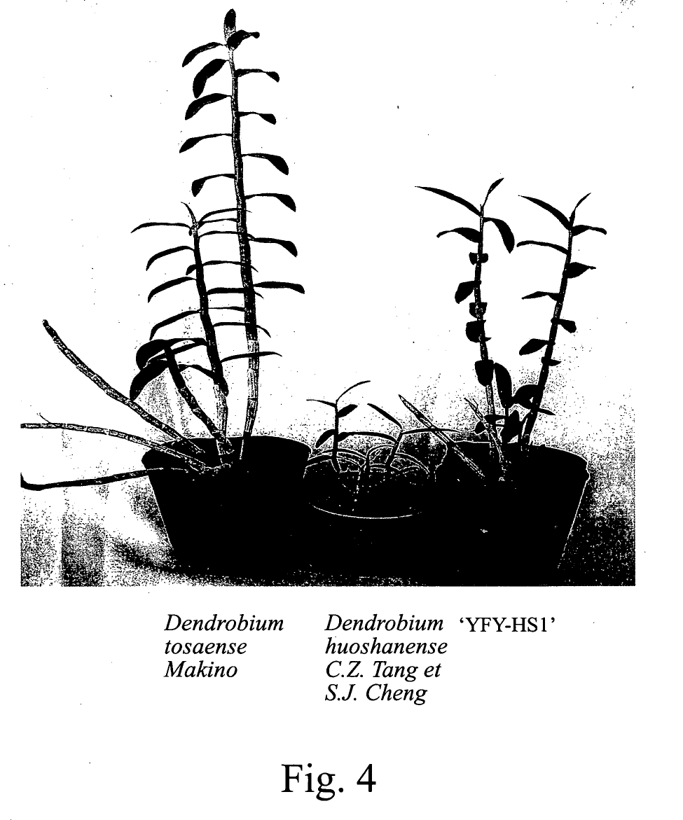

Please refer to FIG. 4, which shows the growth types of the mature D. huoshanese C. Z. Tang et S. J. Cheng, the matured D. tosaense Makino and the matured “YFY-HS1”. After being cultured for about 18 months, it's found, as shown in FIG. 4, the growth type of the “YFY-HS1” is more similar to that of the D. huoshanese C. Z. Tang et S. J. Cheng than that of the D. tosaense Makino.

Please refer to FIGS. 5-7, which respectively show the HPLC profiles of the D. tosaense Makino, the D. huoshanese C. Z. Tang et S. J. Cheng and the “YFY-HS1”. The relevant analyzing conditions are:
IINSTRUMENT: Waters HPLC (WATERS 600)
COLUMN: VERCORPAK Inertsil 5 ODS-2 4.6×150 mm
FLUENT:
   A 0.1% Acetic acid
   B 100% MeOH
GRADIENT: A/B 80/20->45/55
FLOW RATE: 1-0.6 ml/min
WAVELENGTH: 337 nm
INJECTION: 100 μl

As shown in FIGS. 5-7, the extract of “DCMPbL6, 7D2H2” (the chromatographic peaks appear at about 42 minute) is indeed contained in the D. huoshanese C. Z. Tang et S. J. Cheng and the “YFY-HS1”.

Through the above descriptions, a new and distinct Dendrobium plant named “YFY-HS1” does have been provided.

The chart used in the identification of the colors is PANTONE Professional Color System. The age of the observed plant is 18 months from planting.
Origin: Seedling from a cross between the Dendrobrium huoshanense C. Z. Tang et S. J. Cheng and Dendrobrium tosaense Makino.
Parentage:
   Patrulineal parent.—Dendrobrium huoshanense C. Z. Tang et S. J. Cheng.
   Matrilineal parent.—Dendrobrium tosaense Makino.
Classification: Dendrobium hybrid.
Propagation: Asexual propagation by tissue culture.
Plant:
   Size.—A mature plant is tufted and has a height of approximately 20-35 cm.
   Growth habit.—Preferring a moisture environment, and growing well under 20-30° C.
   Flowers per stem.—Approximately 2 to 5.
   Stems.—Tufted, erect, terete, 20 -35 cm in height, 4-7.5 mm in diameter, somewhat slender towards the base.
Leaf:
   General shape.—Oblong-lanceolate.
   Shape of tip.—Acute to sub-obtuse.
   Direction of leaf.—Alternate.
   Length.—3.5-6 cm.
   Width.—1.2-1.5 cm.
   Surface color.—Green pantone 18-0125, 18-0121, 18-0117, 18-0317.
   Underside color.—Pale green pantone 18-0121, 18-0117.
   Thickness.—0.1-0.2 cm.
   Variegation.—None.
   Color of sheath.—Gray green pantone 14-4501.
Flowers:
   Inflorescence.—Racemes, born on the upper nodes, 2-4 cm in length.
   Flower.—2-2.5 cm in length, 2.5-3.0 cm in width.
   Sepals.—Upper sepal: Ovate-oblong, 1.0-1.4 cm in length, 0.4-0.6 cm in width. Lateral sepal: Obliquely triangular and somewhat sub-obtuse, 1.0-1.4 cm in length, 0.5-0.8 cm in width.
   Petal.—Elliptic, 1.3-1.7 cm in length, 0.6-0.8 cm in width.
   Lip shape.—Somewhat wide-rhombate, 1.3-1.7 cm in length, 1.3-1.7 cm in width, central disc with a yellow fleshy elevation, extending forwards with one transverse violet brown band.
   Color of flow.—Ivory yellow.
   Column.—3.0-5.0 mm in length, 0.8-1.0 cm in width, column-foot curved, with purple spots inside.
   Anther cap.—Ovate.
   Pollon color.—Yellow pantone 13-0758, 13-0752, 13-0742.
   Bloom period.—7-14 days.
   Fragrance.—Delicate fragrance.
   Flowering time.—April to June.
   Pseudobulb.—None.
   Reproduction organs.—Column: 3.0 -5.0 mm in length, 0.8-1.0 cm in width, column-foot curved, with purple spots inside. Pollinia: 4 in numbers; color: yellow pantone 13-0758, 13-0752, 13-0742 Pedicel: 2-3 cm in length.
Disease resistance: no specific resistance.
Insect resistance: no specific resistance.

1. A new and distinct variety of Dendrobium plant named as ‘YFY-HS1’ having the characteristics herein illustrated and described.
